(19) United States
(12) Patent Application Publication
(10) Pub. No.: US 2025/0057739 A1
(43) Pub. Date: Feb. 20, 2025

(54) COMPOSITION COMPRISING HYDROPHOBICIZED CATIONIC POLYMER

(71) Applicant: L'Oreal, Paris (FR)

(72) Inventors: Takehiko Kasai, Kanagawa (JP); Nathalie Boileau-Lete, Chevilly Larue (FR); Toshifumi Shiroya, Kanagawa (JP); Hidehiko Asanuma, Kanagawa (JP); Shinsuke Okuda, Kanagawa (JP); Tatsushi Isojima, Kanagawa (JP); Mariko Yamamoto, Kanagawa (JP)

(73) Assignee: L'Oreal, Paris (FR)

(21) Appl. No.: 18/721,076

(22) PCT Filed: Dec. 9, 2022
(86) PCT No.: PCT/JP2022/046299

§ 371 Date: Jun. 17, 2024

(30) Foreign Application Priority Data
Dec. 20, 2021 (JP) 2021-205990
Jan. 20, 2022 (FR) FR2200463

(51) Int. Cl. A61K 8/06; A61K 8/36; A61K 8/365; A61K 8/73; A61K 8/92; A61Q 19/00

(52) U.S. Cl. CPC A61K 8/062; A61K 8/361; A61K 8/365; A61K 8/736; A61K 8/922; A61Q 19/00; A61K 2800/10; A61K 2800/95

(57) ABSTRACT

The present invention relates to a composition comprising: (a-1) at least one cationic polymer, (a-2) at least one monovalent non-polymeric acid or a salt thereof, (a-3) water, (b-1) at least one oil, and (b-2) at least one fatty acid. The composition according to the present invention is stable even when it comprises a relatively large amount of oil, such as up to 40% by weight relative to the total weight of the composition.

### TECHNICAL FIELD

The present invention relates to a composition including a hydrophobicized cationic polymer, as well as a cosmetic process using the composition.

### BACKGROUND ART

A polyion complex, which is formed with an anionic polymer and a cationic polymer, has already been known.

For example, WO 2021/125069 discloses a composition which is useful for cosmetic treatments and comprises at least one polyion complex particle comprising at least one cationic polymer, at least one anionic polymer and at least one non-polymeric acid having two or more pKa values. WO 2021/125069 also discloses that the composition disclosed therein may include oil and may be in the form of an emulsion.

### DISCLOSURE OF INVENTION

However, the composition disclosed in WO 2021/125069 has been found to be stable only when it includes a very limited amount of oil, such as 0.5% by weight relative to the total weight of the composition. When the composition disclosed therein includes a relatively large amount of oil, it tends to be unstable.

Thus, an objective of the present invention is to provide a composition which comprises a cationic polymer and is stable even when it comprises a relatively large amount of oil.

The above objective of the present invention can be achieved by a composition, comprising:
(a-1) at least one cationic polymer;
(a-2) at least one monovalent non-polymeric acid or a salt thereof;
(a-3) water;
(b-1) at least one oil; and
(b-2) at least one fatty acid.

The (a-1) cationic polymer can be hydrophobicized by the (b-2) fatty acid.

The (a-1) cationic polymer may be selected from the group consisting of cyclopolymers of alkyldiallylamine and cyclopolymers of dialkyldiallylammonium such as (co)polydiallyldialkyl ammonium chloride, (co)polyamines such as (co)polylysines and chitosans, cationic (co)polyaminoacids such as collagen, cationic cellulose polymers, and salts thereof.

The amount of the (a-1) cationic polymer(s) in the composition according to the present invention may be from 0.01% to 15% by weight, preferably from 0.05% to 10% by weight, and more preferably from 0.1% to 5% by weight, relative to the total weight of the composition.

The (a-2) monovalent non-polymeric acid may be a monovalent non-polymeric organic acid, preferably a monovalent carboxylic acid, and more preferably lactic acid.

The amount of the (a-2) monovalent non-polymeric acid or a salt thereof in the composition according to the present invention may be from 0.01% to 20% by weight, preferably from 0.05% to 15% by weight, and more preferably from 0.1% to 10% by weight, relative to the total weight of the composition.

The amount of the (a-3) water in the composition according to the present invention may be from 40% to 90% by weight, preferably from 45% to 85% by weight, and more preferably from 50% to 80% by weight, relative to the total weight of the composition.

The (b-1) oil may be selected from plant oils, synthetic ester oils, mixtures thereof, and preferably from plant oils.

The amount of the (b-1) oil(s) in the composition according to the present invention may be from 1% to 50% by weight, preferably from 10% to 45% by weight, and more preferably from 20% to 40% by weight, relative to the total weight of the composition.

The (b-2) fatty acid may be selected from C₄-C₂₂, preferably C₆-C₂₀, more preferably C₈-C₁₈ saturated and unsaturated, linear or branched fatty acids.

The amount of the (b-2) fatty acid(s) in the composition according to the present invention may be from 0.01% to 15% by weight, preferably from 0.05% to 10% by weight, and more preferably from 0.1% to 5% by weight, relative to the total weight of the composition.

The composition according to the present invention may comprise an (a) aqueous phase comprising the (a-1) cationic polymer, the (a-2) monovalent non-polymeric acid or a salt thereof, and the (a-3) water.

The composition according to the present invention may comprise (b) fatty phases comprising the (b-1) oil, and the (b-2) fatty acid.

The present invention also relates to a cosmetic process for a keratin substance such as skin, comprising:
applying to the keratin substance the composition according to the present invention; and drying the composition to form a cosmetic film on the keratin substance.

The present invention also relates to a use of (b-2) at least one fatty acid in a composition, comprising:
(a-1) at least one cationic polymer,
(a-2) at least one monovalent non-polymeric acid or a salt thereof,
(a-3) water, and
(b-1) at least one oil
   in order to increase the amount of the (b-1) oil in the composition to be 1% by weight or more, preferably 10% by weight or more, and more preferably 20% by weight or more, relative to the total weight of the composition.

### BRIEF DESCRIPTION OF DRAWINGS

FIG. 1 shows schematic drawings showing the behaviors of the (a-1) cationic polymers around a (b) fatty phase, such as an oil droplet, dispersed in an (a) aqueous phase, and the examples of hydrophobicization of the (a-1) cationic polymer, in one embodiment of the present invention.

FIG. 1A shows a schematic drawing showing the behaviour of the (a-1) cationic polymers around a (b) fatty phase, such as an oil droplet, dispersed in an (a) aqueous phase, in case of the (b) fatty phase including no (b-2) fatty acid.

FIG. 1B shows a schematic drawing showing the behaviour of the (a-1) cationic polymers around a (b) fatty phase, such as an oil droplet, dispersed in an (a) aqueous phase, in case of the (b) fatty phase including (b-2) fatty acid.

FIG. 1C shows a schematic drawing showing the example of mechanism of hydrophobicization of the (a-1) cationic polymer by the (b-2) fatty acid.

FIG. 2 shows photomicrographs of the compositions according to Example 1 and Comparative Example 1.

### BEST MODE FOR CARRYING OUT THE INVENTION

After diligent research, the inventors have discovered that it is possible to provide a composition which comprises a cationic polymer and is stable even when it comprises a relatively large amount of oil. Thus, the composition according to the present invention comprises:
(a-1) at least one cationic polymer;
(a-2) at least one monovalent non-polymeric acid or a salt thereof;
(a-3) water;
(b-1) at least one oil; and
(b-2) at least one fatty acid.

The composition according to the present invention may comprise an (a) aqueous phase comprising the (a-1) cationic polymer, the (a-2) monovalent non-polymeric acid or a salt thereof, and the (a-3) water.

The composition according to the present invention may comprise (b) fatty phases comprising the (b-1) oil, and the (b-2) fatty acid.

The fatty phases (b) may be dispersed in the (a) aqueous phase.

The (a-1) cationic polymer can be hydrophobicized by the (b-2) fatty acid. Thus, the (b) fatty phases can be stably dispersed in the (a) aqueous phase.

The composition according to the present invention is stable even when it comprises a relatively large amount of oil, such as up to 40% by weight relative to the total weight of the composition. Thus, the composition according to the present invention can comprise a relatively large amount of oil such as up to 40% by weight relative to the total weight of the composition.

The composition according to the present invention is stable for a long period of time. In other words, the phase separation of the composition according to the present invention can be prevented for a long period of time.

Accordingly, the composition according to the present invention can be stored for a long period of time.

Hereinafter, the present invention will be explained in a more detailed manner.

### [Composition]

The composition according to the present invention may comprise:
(a) an aqueous phase; and
(b) fatty phases,
   wherein
   the (b) fatty phases can be dispersed in the (a) aqueous phase.

Thus, the (a) aqueous phase can function as a continuous phase, while the (b) fatty phases can function as discontinuous phases. The composition according to the present invention may be in the form of an O/W dispersion such as an O/W emulsion. As the composition according to the present invention can be of the O/W type, it can provide a fresh sensation due to the aqueous phase including water which forms the outer phase thereof.

The (a) aqueous phase may comprise (a-1) at least one cationic polymer, (a-2) at least one monovalent non-polymeric acid or a salt thereof, and (a-3) water.

The amount of the (a) aqueous phase in the composition according to the present invention may be 45% by weight or more, preferably 50% by weight or more, and more preferably 55% by weight or more, relative to the total weight of the composition.

On the other hand, the amount of the (a) aqueous phase in the composition according to the present invention may be 95% by weight or less, preferably 90% by weight or less, and more preferably 85% by weight or less, relative to the total weight of the composition.

Accordingly, the amount of the (a) aqueous phase in the composition according to the present invention may range from 45% to 95% by weight, preferably from 50% to 90% by weight, and more preferably from 55% to 85% by weight, relative to the total weight of the composition.

The (b) fatty phases may comprise (b-1) at least one oil, and (b-2) at least one fatty acid.

The amount of the (b) fatty phases in the composition according to the present invention may be 5% by weight or more, preferably 15% by weight or more, and more preferably 25% by weight or more, relative to the total weight of the composition.

On the other hand, the amount of the (b) fatty phases in the composition according to the present invention may be 55% by weight or less, preferably 50% by weight or less, and more preferably 45% by weight or less, relative to the total weight of the composition.

Accordingly, the amount of the (b) fatty phases in the composition according to the present invention may range from 5% to 55% by weight, preferably from 15% to 50% by weight, and more preferably from 25% to 45% by weight, relative to the total weight of the composition.

### (Cationic Polymer)

The composition according to the present invention includes (a-1) at least one cationic polymer. A single type of cationic polymer may be used, or two or more different types of cationic polymers may be used in combination.

A cationic polymer has a positive charge density. The charge density of the cationic polymer may be from 0.01 meq/g to 20 meq/g, preferably from 0.05 meq/g to 15 meq/g, and more preferably from 0.1 meq/g to 10 meq/g.

It may be preferable that the molecular weight of the cationic polymer be 1000 or more, preferably 2000 or more, more preferably 3000 or more, and even more preferably 4000 or more.

Unless otherwise defined in the descriptions, “molecular weight” means a number average molecular weight.

The cationic polymer may have at least one positively chargeable and/or positively charged moiety selected from the group consisting of a primary, secondary or tertiary amino group, a quaternary ammonium group, a guanidine group, a biguanide group, an imidazole group, an imino group, and a pyridyl group. The term (primary) “amino group” here means a group of —NH₂.

The cationic polymer may be a homopolymer or a copolymer. The term “copolymer” is understood to mean both copolymers obtained from two kinds of monomers and those obtained from more than two kinds of monomers, such as terpolymers which are in turn obtained from three kinds of monomers.

The cationic polymer may be selected from natural and synthetic cationic polymers, and preferably from natural cationic polymers. Non-limiting examples of the cationic polymers are as follows.

(1) Homopolymers and copolymers derived from acrylic or methacrylic esters and amides and comprising at least one unit chosen from units of the following formulas:

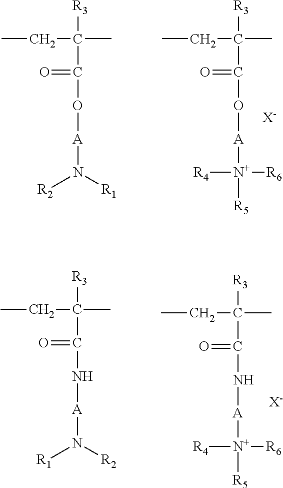

wherein:
R₁ and R₂, which may be identical or different, are chosen from hydrogen and alkyl groups comprising from 1 to 6 carbon atoms, for instance, methyl and ethyl groups;
R₃, which may be identical or different, is chosen from hydrogen and CH₃;
the symbols A, which may be identical or different, are chosen from linear or branched alkyl groups comprising from 1 to 6 carbon atoms, for example, from 2 to 3 carbon atoms and hydroxyalkyl groups comprising from 1 to 4 carbon atoms;
R₄, R₅, and R₆, which may be identical or different, are chosen from alkyl groups comprising from 1 to 18 carbon atoms and benzyl groups, and in at least one embodiment, alkyl groups comprising from 1 to 6 carbon atoms; and
X is an anion derived from an inorganic or organic acid, such as methosulphate anions and halides, for instance chloride and bromide.

The copolymers of family (1) may also comprise at least one unit derived from comonomers which may be chosen from acrylamides, methacrylamides, diacetone acrylamides, acrylamides and methacrylamides substituted on the nitrogen atom with (C₁-C₄) lower alkyl groups, groups derived from acrylic or methacrylic acids and esters thereof, vinyllactams such as vinylpyrrolidone and vinylcaprolactam, and vinyl esters.

Examples of copolymers of family (1) include, but are not limited to:
copolymers of acrylamide and of dimethylaminoethyl methacrylate quaternized with dimethyl sulphate or with a dimethyl halide,
copolymers of acrylamide and of methacryloyloxyethyltrimethylammonium chloride described, for example, in European Patent Application No. 0 080 976,
copolymers of acrylamide and of methacryloyloxyethyltrimethylammonium methosulphate, quaternized or nonquaternized vinylpyrrolidone/dialkylaminoalkyl acrylate or methacrylate copolymers, described, for example, in French Patent Nos. 2 077 143 and 2 393 573, dimethylaminoethyl methacrylate/vinylcaprolactam/vinylpyrrolidone terpolymers, vinylpyrrolidone/methacrylamidopropyldimethylamine copolymers, quaternized vinylpyrrolidone/dimethylaminopropylmethacrylamide copolymers, and
crosslinked methacryloyloxy(C₁-C₄)alkyltri(C₁-C₄)alkylammonium salt polymers such as the polymers obtained by homopolymerization of dimethylaminoethyl methacrylate quaternized with methyl chloride, or by copolymerization of acrylamide with dimethylaminoethyl methacrylate quaternized with methyl chloride, the homopolymerization or copolymerization being followed by crosslinking with a compound containing an olefinic unsaturation, for example, methylenebisacrylamide.
   (2) Cationic cellulose polymers such as cellulose ether derivatives comprising one or more quaternary ammonium groups described, for example, in French Patent No. 1 492 597, such as the polymers sold under the names “JR” (JR 400, JR 125, JR 30M) or “LR” (LR 400, LR 30M) by the company Union Carbide Corporation. These polymers are also defined in the CTFA dictionary as quaternary ammoniums of hydroxyethylcellulose that have reacted with an epoxide substituted with a trimethylammonium group.

It is preferable that the cationic cellulose polymer have at least one quaternary ammonium group, preferably a quaternary trialkyl ammonium group, and more preferably a quaternary trimethyl ammonium group.

The quaternary ammonium group may be present in a quaternary ammonium group-containing group which may be represented by the following chemical formula (I):

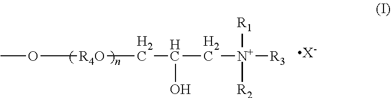

wherein
each of R₁ and R₂ denotes a C₁₋₃ alkyl group, preferably a methyl or ethyl group, and more preferably a methyl group,
R₃ denotes a C₁₋₂₄ alkyl group, preferably a methyl or ethyl group, and more preferably methyl group, X⁻ denotes an anion, preferably a halide, and more preferably a chloride,
n denotes an integer from 0-30, preferably 0-10, and more preferably 0, and
R₄ denotes a C₁₋₄ alkylene group, preferably an ethylene or propylene group.

The leftmost ether bond (—O—) in the above chemical formula (I) can attach to the sugar ring of the polysaccharide.

It is preferable that the quaternary ammonium group-containing group be —O—CH₂—CH(OH)—CH₂—N⁺(CH₃)₃.

(3) Cationic cellulose polymers such as cellulose copolymers and cellulose derivatives grafted with a water-soluble monomer of quaternary ammonium, and described, for example, in U.S. Pat. No. 4,131,576, such as hydroxyalkylcelluloses, for instance, hydroxymethyl-, hydroxyethyl-, and hydroxypropylcelluloses grafted, for example, with a salt chosen from methacryloylethyltrimethylammonium, methacrylamidopropyltrimethylammonium, and dimethyldiallylammonium salts.

Commercial products corresponding to these polymers include, for example, the products sold under the name “Celquat® L 200” and “Celquat® H 100” by the company National Starch.

(4) Non-cellulose-based cationic polysaccharides described in U.S. Pat. Nos. 3,589,578 and 4,031,307, such as guar gums comprising cationic trialkylammonium groups, cationic hyaluronic acid, and dextran hydroxypropyl trimonium chloride. Guar gums modified with a salt, for example the chloride, of 2,3-epoxypropyltrimethylammonium (guar hydroxypropyltrimonium chloride) may also be used.

Such products are sold, for instance, under the trade names JAGUAR® C13 S, JAGUAR® C15, JAGUAR® C17, and JAGUAR® C162 by the company MEYHALL.

(5) Polymers comprising piperazinyl units and divalent alkylene or hydroxyalkylene groups comprising straight or branched chains, optionally interrupted with at least one entity chosen from oxygen, sulphur, nitrogen, aromatic rings, and heterocyclic rings, and also the oxidation and/or quaternization products of these polymers. Such polymers are described, for example, in French Patent Nos. 2 162 025 and 2 280 361.
(6) Water-soluble polyamino amides prepared, for example, by polycondensation of an acidic compound with a polyamine; these polyamino amides possibly being crosslinked with an entity chosen from epihalohydrins; diepoxides; dianhydrides; unsaturated dianhydrides; bisunsaturated derivatives; bishalohydrins; bisazetidiniums; bishaloacyidiamines; bisalkyl halides; oligomers resulting from the reaction of a difunctional compound which is reactive with an entity chosen from bishalohydrins, bisazetidiniums, bishaloacyldiamines, bisalkyl halides, epihalohydrins, diepoxides, and bisunsaturated derivatives; the crosslinking agent being used in an amount ranging from 0.025 to 0.35 mol per amine group of the polyamino amide; these polyamino amides optionally being alkylated or, if they comprise at least one tertiary amine function, they may be quatemized. Such polymers are described, for example, in French Patent Nos. 2 252 840 and 2 368 508.
(7) Polyamino amide derivatives resulting from the condensation of polyalkylene polyamines with polycarboxylic acids, followed by alkylation with difunctional agents, for example, adipic acid/dialkylaminohydroxyalkyldialkylenetriamine polymers in which the alkyl group comprises from 1 to 4 carbon atoms, such as methyl, ethyl, and propyl groups, and the alkylene group comprises from 1 to 4 carbon atoms, such as an ethylene group. Such polymers are described, for instance, in French Patent No. 1 583 363. In at least one embodiment, these derivatives may be chosen from adipic acid/dimethylaminohydroxypropyldiethylenetriamine polymers.
(8) Polymers obtained by reaction of a polyalkylene polyamine comprising two primary amine groups and at least one secondary amine group, with a dicarboxylic acid chosen from diglycolic acid and saturated aliphatic dicarboxylic acids comprising from 3 to 8 carbon atoms. The molar ratio of the polyalkylene polyamine to the dicarboxylic acid may range from 0.8:1 to 1.4:1; the polyamino amide resulting therefrom being reacted with epichlorohydrin in a molar ratio of epichlorohydrin relative to the secondary amine group of the polyamino amide ranging from 0.5:1 to 1.8:1. Such polymers are described, for example, in U.S. Pat. Nos. 3,227,615 and 2,961,347.
(9) Cyclopolymers of alkyldiallylamine and cyclopolymers of dialkyldiallyl-ammonium, such as homopolymers and copolymers comprising, as the main constituent of the chain, at least one unit chosen from units of formulas (Ia) and (Ib):

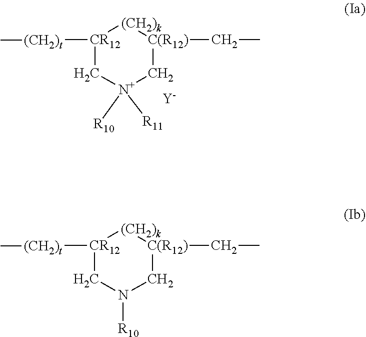

wherein:
k and t, which may be identical or different, are equal to 0 or 1, the sum k+t being equal to 1;
R₁₂ is chosen from hydrogen and methyl groups;
R₁₀ and R₁₁, which may be identical or different, are chosen from alkyl groups comprising from 1 to 6 carbon atoms, hydroxyalkyl groups in which the alkyl group comprises, for example, from 1 to 5 carbon atoms, and lower (C₁-C₄)amidoalkyl groups, or R₁₀ and R₁₁ may form, together with the nitrogen atom to which they are attached, heterocyclic groups such as piperidinyl and morpholinyl; and
Y′ is an anion such as bromide, chloride, acetate, borate, citrate, tartrate, bisulphate, bisulphite, sulphate, and phosphate. These polymers are described, for example, in French Patent No. 2 080 759 and in its Certificate of Addition 2 190 406.

In one embodiment, R₁₀ and R₁₁, which may be identical or different, are chosen from alkyl groups comprising from 1 to 4 carbon atoms.

Examples of such polymers include, but are not limited to, (co)polydiallyldialkyl ammonium chloride such as the dimethyidiallylammonium chloride homopolymer sold under the name “MERQUAT® 100” by the company CALGON (and its homologues of low weight-average molecular mass) and the copolymers of diallyldimethylammonium chloride and of acrylamide sold under the name “MERQUAT® 550”.

Quaternary diammonium polymers comprising at least one repeating unit of formula (II):

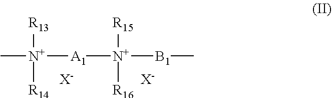

wherein:
R₁₃, R₁₄, R₁₅, and R₁₆, which may be identical or different, are chosen from aliphatic, alicyclic, and arylaliphatic groups comprising from 1 to 20 carbon atoms and lower hydroxyalkyl aliphatic groups, or alternatively R₁₃, R₁₄, R₁₅, and R₁₆ may form, together or separately, with the nitrogen atoms to which they are attached, heterocycles optionally comprising a second heteroatom other than nitrogen, or alternatively R₁₃, R₁₄, R₁₅, and R₁₆, which may be identical or different, are chosen from linear or branched C₁-C₆ alkyl groups substituted with at least one group chosen from nitrile groups, ester groups, acyl groups, amide groups, —CO—O—R₁₇-E groups, and —CO—NH—R₁₇-E groups, wherein R₁₇ is an alkylene group and E is a quaternary ammonium group;
A₁ and B₁, which may be identical or different, are chosen from polymethylene groups comprising from 2 to 20 carbon atoms, which may be linear or branched, saturated or unsaturated, and which may comprise, linked or intercalated in the main chain, at least one entity chosen from aromatic rings, oxygen, sulphur, sulphoxide groups, sulphone groups, disulphide groups, amino groups, alkylamino groups, hydroxyl groups, quaternary ammonium groups, ureido groups, amide groups, and ester groups, and
X⁻ is an anion derived from an inorganic or organic acid;
A₁, R₁₃, and R₁₅ may form, together with the two nitrogen atoms to which they are attached, a piperazine ring;
if A₁ is chosen from linear or branched, saturated or unsaturated alkylene or hydroxyalkylene groups, B₁ may be chosen from:

—(CH₂)ₙ—CO-E′-OC—(CH₂)ₙ—

wherein E′ is chosen from:
a) glycol residues of formula —O—Z—O—, wherein Z is chosen from linear or branched hydrocarbon-based groups and groups of the following formulas:

—(CH₂—CH₂—O)_{X}—CH₂—CH₂—

—[CH₂—CH(CH₃)—O]_{y}—CH₂—CH(CH₃)—

wherein x and y, which may be identical or different, are chosen from integers ranging from 1 to 4, which represent a defined and unique degree of polymerization, and numbers ranging from 1 to 4, which represent an average degree of polymerization;
b) bis-secondary diamine residue such as piperazine derivatives;
c) bis-primary diamine residues of formula —NH—Y—NH—, wherein Y is chosen from linear or branched hydrocarbon-based groups and the divalent group —CH₂—CH₂—S—S—CH₂—CH₂—; and
d) ureylene groups of formula —NH—CO—NH—.

In at least one embodiment, X⁻ is an anion such as chloride or bromide.

Polymers of this type are described, for example, in French Patent Nos. 2 320 330; 2 270 846; 2 316 271; 2 336 434; and 2 413 907 and U.S. Pat. Nos. 2,273,780; 2,375,853; 2,388,614; 2,454,547; 3,206,462; 2,261,002; 2,271,378; 3,874,870; 4,001,432; 3,929,990; 3,966,904; 4,005,193; 4,025,617; 4,025,627; 4,025,653; 4,026,945; and 4,027,020.

Non-limiting examples of such polymers include those comprising at least one repeating unit of formula (III):

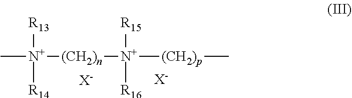

wherein
R₁₃, R₁₄, R₁₅, and R₁₆, which may be identical or different, are chosen from alkyl and hydroxyalkyl groups comprising from 1 to 4 carbon atoms, n and p, which may be identical or different, are integers ranging from 2 to 20, and X⁻ is an anion derived from an inorganic or organic acid.
   (11) Polyquaternary ammonium polymers comprising units of formula (IV):

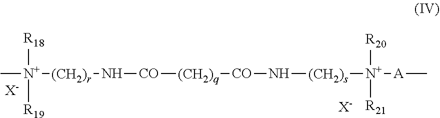

wherein:
R₁₈, R₁₉, R₂₀, and R₂₁, which may be identical or different, are chosen from hydrogen, methyl groups, ethyl groups, propyl groups, β-hydroxyethyl groups, β-hydroxypropyl groups, —CH₂CH₂(OCH₂CH₂)ₚOH groups, wherein p is chosen from integers ranging from 0 to 6, with the proviso that R₁₈, R₁₉, R₂₀, and R₂₁ are not simultaneously hydrogen,
r and s, which may be identical or different, are chosen from integers ranging from 1 to 6,
q is chosen from integers ranging from 0 to 34,
X⁻ is an anion such as a halide, and
A is chosen from radicals of dihalides and —CH₂—CH₂—O—CH₂—CH₂—.

Such compounds are described, for instance, in European Patent Application No. 0 122 324.

### (12) Quaternary Polymers of Vinylpyrrolidone and of Vinylimidazole.

Other examples of suitable cationic polymers include, but are not limited to, cationic proteins and cationic protein hydrolysates, polyalkyleneimines, such as polyethyleneimines, polymers comprising units chosen from vinylpyridine and vinylpyridinium units, condensates of polyamines and of epichlorohydrin, quaternary polyureylenes, and chitin derivatives.

According to one embodiment of the present invention, the at least one cationic polymer is chosen from cellulose ether derivatives comprising quaternary ammonium groups, such as the products sold under the name “JR 400” by the company UNION CARBIDE CORPORATION, cationic cyclopolymers, for instance, the homo-polymers and copolymers of dimethyldiallylammonium chloride sold under the names MERQUAT® 100, MERQUAT® 550, and MERQUAT® S by the company CALGON, guar gums modified with a 2,3-epoxypropyltrimethylammonium salt, and quaternary polymers of vinylpyrrolidone and of vinylimidazole.

### (13) Polyamines

As the cationic polymer, it is also possible to use (co)polyamines, which may be homopolymers or copolymers, with a plurality of amino groups. The amino group may be a primary, secondary, tertiary or quaternary amino group. The amino group may be present in a polymer backbone or a pendent group, if present, of the (co)polyamines.

As an example of the (co)polyamines, mention may be made of chitosan, (co)polyallylamines, (co)polyvinylamines, (co)polyanilines, (co)polyvinylimidazoles, (co)polydimethylaminoethylenemethacrylates, (co)polyvinylpyridines such as (co)poly-1-methyl-2-vinylpyridines, (co)polyimines such as (co) polyethyleneimines, (co)polypyridines such as (co)poly(quaternary pyridines), (co)polybiguanides such as (co)polyaminopropyl biguanides, (co)polylysines, (co)polyomithines, (co)polyarginines, (co)polyhistidines, aminodextrans, aminocelluloses, amino(co)polyvinylacetals, and salts thereof.

As the (co)polyamines, it is preferable to use chitosans. Chitosan is well known. Chitosan can be a linear polysaccharide composed of randomly distributed β-(1→4)-linked D-glucosamine (deacetylated unit) and N-acetyl-D-glucosamine (acetylated unit). The molecular weight of chitosan may be from 5,000 to 1,000,000, preferably from 10,000 to 500,000, and more preferably from 50,000 to 200,000. The actylation degree of chitosan may be from 1.0% to 10.0%, preferably from 2.0% to 8.0%, and more preferably from 3.0% to 6.0%. Chitosan can be prepared by, for example, treating the chitin shells of shrimp and other crustaceans with an alkaline substance, such as sodium hydroxide.

As the (co)polyamines, it may be preferable to use (co)polylysines. Polylysine is also well known. Polylysine can be a natural homopolymer of L-lysine that can be produced by bacterial fermentation. For example, polylysine can be ε-Poly-L-lysine, typically used as a natural preservative in food products. Polylysine is a polyelectrolyte which is soluble in polar solvents such as water, propylene glycol and glycerol. Polylysine is commercially available in various forms, such as poly D-lysine and poly L-lysine. Polylysine can be in salt and/or solution form.

### (14) Cationic Polyaminoacids

As the cationic polymer, it may be possible use cationic polyaminoacids, which may be cationic homopolymers or copolymers, with a plurality of amino groups and carboxyl groups. The amino group may be a primary, secondary, tertiary or quaternary amino group. The amino group may be present in a polymer backbone or a pendent group, if present, of the cationic polyaminoacids. The carboxyl group may be present in a pendent group, if present, of the cationic polyaminoacids.

As examples of the cationic polyaminoacids, mention may be made of cationized collagen, cationized gelatin, steardimoium hydroxyprolyl hydrolyzed wheat protein, cocodimonium hydroxypropyl hydrolyzed wheat protein, hydroxypropyltrimonium hydrolyzed conchiolin protein, steardimonium hydroxypropyl hydrolyzed soy protein, hydroxypropyltrimonium hydrolyzed soy protein, cocodimonium hydroxypropyl hydrolyzed soy protein, and the like.

The following descriptions relate to preferable embodiments of the cationic polymer.

It may be preferable that the (a-1) cationic polymer be selected from cationic starches.

As examples of the cationic starches, mention may be made of starches modified with a 2,3-epoxypropyltrimethylammonium salt (e.g. chloride), such as the product known as starch hydroxypropyltrimonium chloride according to the INCl nomenclature and sold under the name SENSOMER™ Cl-50 from Ondeo or Pencare™ DP 1015 from Ingredion.

It may also be preferable that the (a-1) cationic polymer be selected from cationic gums.

The gums may be, for example, selected from the group consisting of cassia gum, karaya gum, konjac gum, gum tragacanth, tara gum, acacia gum and gum arabic.

Examples of cationic gum include cationic polygalactomannan derivatives such as guar gum derivatives and cassia gum derivatives, e.g., CTFA: Guar Hydroxypropyltrimonium Chloride, Hydroxypropyl Guar Hydroxypropyltrimonium Chloride, and Cassia Hydroxypropyltrimonium Chloride. Guar hydroxypropyltrimonium chloride is commercially available under the Jaguar™ trade name series from Rhodia Inc. and the N-Hance trade name series from Ashland Inc. Cassia Hydroxypropyltrimonium Chloride is commercially available under the Sensomer™ CT-250 and Sensomer™ CT-400 trademarks from Lubrizol Advanced Materials, Inc or the ClearHance™ from Ashland Inc.

It may also be preferable that the (a-1) cationic polymer be selected from chitosans.

It may be more preferable that the (a-1) cationic polymer be selected from the group consisting of cyclopolymers of alkyldiallylamine and cyclopolymers of dialkyldiallylammonium such as (co)polydiallyldialkyl ammonium chloride, (co)polyamines such as (co)polylysines and chitosans, cationic (co)polyaminoacids such as cationized collagen, cationic cellulose polymers, and salts thereof.

It may be even more preferable that the (a-1) cationic polymer be selected from the group consisting of polyquaternium-4, polyquaternium-10, polyquatemium-24, polyquaternium-67, starch hydroxypropyl trimonium chloride, cassia hydroxypropyltrimonium chloride, polylysine, chitosan, and a mixture thereof.

The amount of the (a-1) cationic polymer(s) in the composition according to the present invention may be 0.01% by weight or more, preferably 0.05% by weight or more, and more preferably 0.1% by weight or more, relative to the total weight of the composition.

The amount of the cationic polymer(s) in the composition according to the present invention may be 15% by weight or less, preferably 10% by weight or less, and more preferably 5% by weight or less, relative to the total weight of the composition.

The amount of the cationic polymer(s) in the composition according to the present invention may be from 0.01% to 15% by weight, preferably from 0.05% to 10% by weight, and more preferably from 0.1% to 5% by weight, relative to the total weight of the composition.

### (Monovalent Non-Polymeric Acid or Salt Thereof)

The composition according to the present invention comprises (a-2) at least one monovalent non-polymeric acid or a salt thereof. Two or more monovalent non-polymeric acid or salts thereof may be used in combination. Thus, a single type of monovalent non-polymeric acid or a salt thereof or a combination of different types of monovalent non-polymeric acids or salts thereof may be used.

The term “non-polymeric” here means that the acid is not obtained by polymerizing two or more monomers. Therefore, the non-polymeric acid does not correspond to an acid obtained by polymerizing two or more monomers such as polyacrylic acids.

The term “salt” here means a salt formed by addition of suitable base(s) to the monovalent non-polymeric acid, which may be obtained from a reaction with the monovalent non-polymeric acid with the base(s) according to methods known to those skilled in the art. As the salt, mention may be made of metal salts, for example salts with alkaline metal such as Na and K, and salts with alkaline earth metal such as Mg and Ca, and ammonium salts.

It is preferable that the molecular weight of the (a-2) monovalent non-polymeric acid or salt thereof be less than 1000, preferably 500 or less, and more preferably 100 or less.

The (a-2) monovalent non-polymeric acid or a salt thereof can be included in the (a) aqueous phase. The (a-2) monovalent non-polymeric acid or a salt thereof may make the (a-1) cationic polymer be dissolved in the (a) aqueous phase. The (a-2) monovalent non-polymeric acid or a salt thereof may form a complex with the (a-1) cationic polymer.

The (a-2) monovalent non-polymeric acid or a salt thereof may be selected from monovalent organic or inorganic acids and salts thereof, preferably monovalent organic acids and salts thereof, and more preferably monovalent carboxylic acids and salts thereof.

The (a-2) monovalent non-polymeric acid has a single acid group which may be selected from the group consisting of a carboxylic group, a sulfuric group, a sulfonic group, a phosphoric group, a phosphonic group, and a mixture thereof.

The (a-2) monovalent non-polymeric acid may be selected from hydroxyl acids, and preferably alpha-hydroxy acids. As the alpha-hydroxy acids, mention may be made of, for example, lactic acid and glycolic acid.

The (a-2) monovalent non-polymeric acid may be a monovalent non-polymeric organic acid, preferably a monovalent carboxylic acid, and more preferably lactic acid.

The amount of the (a-2) monovalent non-polymeric acid(s) or a salt thereof in the composition according to the present invention may be 0.01% by weight or more, preferably 0.05% by weight or more, and more preferably 0.1% by weight or more, relative to the total weight of the composition.

The amount of the (a-2) monovalent non-polymeric acid(s) or a salt thereof in the composition according to the present invention may be 15% by weight or less, preferably 10% by weight or less, and more preferably 5% by weight or less, relative to the total weight of the composition.

The amount of the (a-2) monovalent non-polymeric acid(s) or a salt thereof in the composition according to the present invention may be from 0.01% to 15% by weight, preferably from 0.05% to 10% by weight, and more preferably from 0.1% to 5% by weight, relative to the total weight of the composition.

### (Water)

The composition according to the present invention comprises (a-3) water.

The (a-3) water can form an aqueous phase which is a continuous phase of the composition according to the present invention.

The amount of the (a-3) water may be 40% by weight or more, preferably 45% by weight or more, and more preferably 50% by weight or more, relative to the total weight of the composition.

The amount of the (a-3) water may be 90% by weight or less, preferably 85% by weight or less, and more preferably 80% by weight or less, relative to the total weight of the composition.

The amount of the (a-3) water may be from 40% to 90% by weight, preferably from 45% to 85% by weight, and more preferably from 50% to 80% by weight, relative to the total weight of the composition.

### (Oil)

The composition according to the present invention comprises (b-1) at least one oil. If two or more (b) oils are used, they may be the same or different.

Here, “oil” means a fatty compound or substance which is in the form of a liquid or a paste (non-solid) at room temperature (25° C.) under atmospheric pressure (760 mmHg). As the oils, those generally used in cosmetics can be used alone or in combination thereof. These oils may be volatile or non-volatile.

The oil may be a non-polar oil such as a hydrocarbon oil, a silicone oil, or the like; a polar oil such as a plant or animal oil and an ester oil or an ether oil; or a mixture thereof.

The oil may be selected from the group consisting of oils of plant or animal origin, synthetic oils, silicone oils, hydrocarbon oils and fatty alcohols.

As examples of plant oils, mention may be made of, for example, apricot oil, linseed oil, camellia oil, macadamia nut oil, corn oil, mink oil, olive oil, avocado oil, sasanqua oil, castor oil, safflower oil, jojoba oil, sunflower oil, almond oil, rapeseed oil, sesame oil, soybean oil, peanut oil, and mixtures thereof.

As examples of animal oils, mention may be made of, for example, squalene and squalane.

As examples of synthetic oils, mention may be made of alkane oils such as isododecane and isohexadecane, ester oils, ether oils, and artificial triglycerides.

The ester oils are preferably liquid esters of saturated or unsaturated, linear or branched C₁-C₂₆ aliphatic monoacids or polyacids and of saturated or unsaturated, linear or branched C₁-C₂₆ aliphatic monoalcohols or polyalcohols, the total number of carbon atoms of the esters being greater than or equal to 10.

Preferably, for the esters of monoalcohols, at least one from among the alcohol and the acid from which the esters of the present invention are derived is branched.

Among the monoesters of monoacids and of monoalcohols, mention may be made of ethyl palmitate, ethyl hexyl palmitate, isopropyl palmitate, dicaprylyl carbonate, alkyl myristates such as isopropyl myristate or ethyl myristate, isocetyl stearate, 2-ethylhexyl isononanoate, isononyl isononanoate, isodecyl neopentanoate and isostearyl neopentanoate.

Esters of C₄-C₂₂ dicarboxylic or tricarboxylic acids and of C₁-C₂₂ alcohols, and esters of monocarboxylic, dicarboxylic or tricarboxylic acids and of non-sugar C₄-C₂₆ dihydroxy, trihydroxy, tetrahydroxy or pentahydroxy alcohols may also be used.

Mention may especially be made of: diethyl sebacate; isopropyl lauroyl sarcosinate; diisopropyl sebacate; bis(2-ethylhexyl) sebacate; diisopropyl adipate; di-n-propyl adipate; dioctyl adipate; bis(2-ethylhexyl) adipate; diisostearyl adipate; bis(2-ethylhexyl) maleate; triisopropyl citrate; triisocetyl citrate; triisostearyl citrate; glyceryl trilactate; glyceryl trioctanoate; trioctyldodecyl citrate; trioleyl citrate; neopentyl glycol diheptanoate; and diethylene glycol diisononanoate.

As ester oils, one can use sugar esters and diesters of C₆-C₃₀ and preferably C₁₂-C₂₂ fatty acids. It is recalled that the term “sugar” means oxygen-bearing hydrocarbon-based compounds containing several alcohol functions, with or without aldehyde or ketone functions, and which comprise at least 4 carbon atoms. These sugars may be monosaccharides, oligosaccharides or polysaccharides.

Examples of suitable sugars that may be mentioned include sucrose (or saccharose), glucose, galactose, ribose, fucose, maltose, fructose, mannose, arabinose, xylose and lactose, and derivatives thereof, especially alkyl derivatives, such as methyl derivatives, for instance methylglucose.

The sugar esters of fatty acids may be chosen especially from the group comprising the esters or mixtures of esters of sugars described previously and of linear or branched, saturated or unsaturated C₆-C₃₀ and preferably C₁₂-C₂₂ fatty acids. If they are unsaturated, these compounds may have one to three conjugated or non-conjugated carbon-carbon double bonds. The esters according to this variant may also be selected from monoesters, diesters, triesters, tetraesters and polyesters, and mixtures thereof.

These esters may be, for example, oleates, laurates, palmitates, myristates, behenates, cocoates, stearates, linoleates, linolenates, caprates and arachidonates, or mixtures thereof such as, especially, oleopalmitate, oleostearate and palmitostearate mixed esters, as well as pentaerythrityl tetraethyl hexanoate.

More particularly, use is made of monoesters and diesters and especially sucrose, glucose or methylglucose monooleates or dioleates, stearates, behenates, oleopalmitates, linoleates, linolenates and oleostearates.

An example that may be mentioned is the product sold under the name Glucate® DO by the company Amerchol, which is a methylglucose dioleate.

As examples of preferable ester oils, mention may be made of, for example, diisopropyl adipate, dioctyl adipate, 2-ethylhexyl hexanoate, ethyl laurate, cetyl octanoate, octyldodecyl octanoate, isodecyl neopentanoate, myristyl propionate, 2-ethylhexyl 2-ethylhexanoate, 2-ethylhexyl octanoate, 2-ethylhexyl caprylate/caprate, methyl palmitate, ethyl palmitate, isopropyl palmitate, dicaprylyl carbonate, isopropyl lauroyl sarcosinate, isononyl isononanoate, ethylhexyl palmitate, isohexyl laurate, hexyl laurate, isocetyl stearate, isopropyl isostearate, isopropyl myristate, isodecyl oleate, glyceryl tri(2-ethylhexanoate), pentaerythrityl tetra(2-ethylhexanoate), 2-ethylhexyl succinate, diethyl sebacate, and mixtures thereof.

As examples of artificial triglycerides, mention may be made of, for example, capryl caprylyl glycerides, glyceryl trimyristate, glyceryl tripalmitate, glyceryl trilinolenate, glyceryl trilaurate, glyceryl tricaprate, glyceryl tricaprylate, glyceryl tri(caprate/caprylate) and glyceryl tri(caprate/caprylate/linolenate).

As examples of silicone oils, mention may be made of, for example, linear organopolysiloxanes such as dimethylpolysiloxane, methylphenylpolysiloxane, methylhydrogenpolysiloxane, and the like; cyclic organopolysiloxanes such as cyclohexasiloxane, octamethylcyclotetrasiloxane, decamethylcyclopentasiloxane, dodecamethylcyclohexasiloxane, and the like; and mixtures thereof.

Preferably, silicone oil is chosen from liquid polydialkylsiloxanes, especially liquid polydimethylsiloxanes (PDMS) and liquid polyorganosiloxanes comprising at least one aryl group.

These silicone oils may also be organomodified. The organomodified silicones that can be used according to the present invention are silicone oils as defined above and comprise in their structure one or more organofunctional groups attached via a hydrocarbon-based group.

Organopolysiloxanes are defined in greater detail in Walter Noll's Chemistry and Technology of Silicones (1968), Academic Press. They may be volatile or non-volatile.

When they are volatile, the silicones are more particularly chosen from those having a boiling point of between 60° C. and 260° C., and even more particularly from:

(i) Cyclic polydialkylsiloxanes comprising from 3 to 7 and preferably 4 to 5 silicon atoms. These are, for example, octamethylcyclotetrasiloxane sold in particular under the name Volatile Silicone® 7207 by Union Carbide or Silbione® 70045 V2 by Rhodia, decamethylcyclopentasiloxane sold under the name Volatile Silicone® 7158 by Union Carbide, Silbione® 70045 V5 by Rhodia, and dodecamethylcyclopentasiloxane sold under the name Silsoft 1217 by Momentive Performance Materials, and mixtures thereof. Mention may also be made of cyclocopolymers of the type such as dimethylsiloxane/methylalkylsiloxane, such as Silicone Volatile® FZ 3109 sold by the company Union Carbide, of formula:

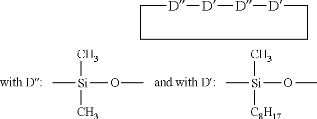

Mention may also be made of mixtures of cyclic polydialkylsiloxanes with organosilicon compounds, such as the mixture of octamethylcyclotetrasiloxane and tetratrimethylsilylpentaerythritol (50/50) and the mixture of octamethylcyclotetrasiloxane and oxy-1,1′-bis(2,2,2′,2′,3,3′-hexatrimethylsilyloxy)neopentane; and

(ii) Linear volatile polydialkylsiloxanes containing 2 to 9 silicon atoms and having a viscosity of less than or equal to 5×10⁻⁶ m²/s at 25° C. An example is decamethyltetrasiloxane sold in particular under the name SH 200 by the company Toray Silicone. Silicones belonging to this category are also described in the article published in Cosmetics and Toiletries, Vol. 91, January 76, pp. 27-32, Todd & Byers, Volatile Silicone Fluids for Cosmetics. The viscosity of the silicones is measured at 25° C. according to ASTM standard 445 Appendix C.

Non-volatile polydialkylsiloxanes may also be used. These non-volatile silicones are more particularly chosen from polydialkylsiloxanes, among which mention may be made mainly of polydimethylsiloxanes containing trimethylsilyl end groups.

Among these polydialkylsiloxanes, mention may be made, in a non-limiting manner, of the following commercial products:
the Silbione® oils of the 47 and 70 047 series or the Mirasil® oils sold by Rhodia, for instance the oil 70 047 V 500 000;
the oils of the Mirasil® series sold by the company Rhodia;
the oils of the 200 series from the company Dow Corning, such as DC200 with a viscosity of 60,000 mm²/s; and
the Viscasil® oils from General Electric and certain oils of the SF series (SF 96, SF 18) from General Electric.

Mention may also be made of polydimethylsiloxanes containing dimethylsilanol end groups known under the name dimethiconol (CTFA), such as the oils of the 48 series from the company Rhodia.

Among the silicones containing aryl groups, mention may be made of polydiarylsiloxanes, especially polydiphenylsiloxanes and polyalkylarylsiloxanes such as phenyl silicone oil.

The phenyl silicone oil may be chosen from the phenyl silicones of the following formula:

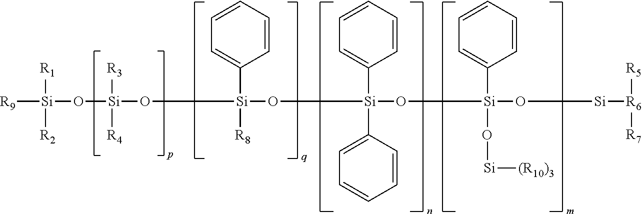

in which
R₁ to R₁₀, independently of each other, are saturated or unsaturated, linear, cyclic or branched C₁-C₃₀ hydrocarbon-based radicals, preferably C₁-C₁₂ hydrocarbon-based radicals, and more preferably C₁-C₆ hydrocarbon-based radicals, in particular methyl, ethyl, propyl or butyl radicals, and
m, n, p and q are, independently of each other, integers 0 to 900 inclusive, preferably 0 to 500 inclusive, and more preferably 0 to 100 inclusive,
with the proviso that the sum n+m+q is not 0.

Examples that may be mentioned include the products sold under the following names:
the Silbione® oils of the 70 641 series from Rhodia;
the oils of the Rhodorsil® 70 633 and 763 series from Rhodia;
the oil Dow Corning 556 Cosmetic Grade Fluid from Dow Corning;
the silicones of the PK series from Bayer, such as the product PK20;
certain oils of the SF series from General Electric, such as SF 1023, SF 1154, SF 1250 and SF 1265.

As the phenyl silicone oil, phenyl trimethicone (R₁ to R₁₀ are methyl; p, q, and n=0; m=1 in the above formula) is preferable.

The organomodified liquid silicones may especially contain polyethyleneoxy and/or polypropyleneoxy groups. Mention may thus be made of the silicone KF-6017 proposed by Shin-Etsu, and the oils Silwet® L722 and L77 from the company Union Carbide.

Hydrocarbon oils may be chosen from:
linear or branched, optionally cyclic, C₆-C₁₆ lower alkanes. Examples that may be mentioned include hexane, undecane, dodecane, tridecane, and isoparaffins, for instance isohexadecane, isododecane and isodecane; and
linear or branched hydrocarbons containing more than 16 carbon atoms, such as liquid paraffins, liquid petroleum jelly, polydecenes and hydrogenated polyisobutenes such as Parleam®, and squalane.

As preferable examples of hydrocarbon oils, mention may be made of, for example, linear or branched hydrocarbons such as isohexadecane, isododecane, squalane, mineral oil (e.g., liquid paraffin), paraffin, vaseline or petrolatum, naphthalenes, and the like; hydrogenated polyisobutene, isoeicosan, and decene/butene copolymer; and mixtures thereof.

The term “fatty” in the fatty alcohol means the inclusion of a relatively large number of carbon atoms. Thus, alcohols which have 4 or more, preferably 6 or more, and more preferably 12 or more carbon atoms are encompassed within the scope of fatty alcohols. The fatty alcohol may be saturated or unsaturated. The fatty alcohol may be linear or branched.

The fatty alcohol may have the structure R—OH wherein R is chosen from saturated and unsaturated, linear and branched radicals containing from 4 to 40 carbon atoms, preferably from 6 to 30 carbon atoms, and more preferably from 12 to 20 carbon atoms. In at least one embodiment, R may be chosen from C₁₂-C₂₀ alkyl and C₁₂-C₂₀ alkenyl groups. R may or may not be substituted with at least one hydroxyl group.

As examples of the fatty alcohol, mention may be made of lauryl alcohol, cetyl alcohol, stearyl alcohol, isostearyl alcohol, behenyl alcohol, undecylenyl alcohol, myristyl alcohol, octyldodecanol, hexyldecanol, oleyl alcohol, linoleyl alcohol, palmitoleyl alcohol, arachidonyl alcohol, erucyl alcohol, and mixtures thereof.

It is preferable that the fatty alcohol be a saturated fatty alcohol.

Thus, the fatty alcohol may be selected from straight or branched, saturated or unsaturated C₆-C₃₀ alcohols, preferably straight or branched, saturated C₆-C₃₀ alcohols, and more preferably straight or branched, saturated C₁₂-C₂₀ alcohols.

The term “saturated fatty alcohol” here means an alcohol having a long aliphatic saturated carbon chain. It is preferable that the saturated fatty alcohol be selected from any linear or branched, saturated C₆-C₃₀ fatty alcohols. Among the linear or branched, saturated C₆-C₃₀ fatty alcohols, linear or branched, saturated C₁₂-C₂₀ fatty alcohols may preferably be used. Any linear or branched, saturated C₁₆-C₂₀ fatty alcohols may be more preferably used. Branched C₁₆-C₂₀ fatty alcohols may be even more preferably used.

As examples of saturated fatty alcohols, mention may be made of lauryl alcohol, cetyl alcohol, stearyl alcohol, isostearyl alcohol, behenyl alcohol, undecylenyl alcohol, myristyl alcohol, octyldodecanol, hexyldecanol, and mixtures thereof. In one embodiment, cetyl alcohol, stearyl alcohol, octyldodecanol, hexyldecanol, or a mixture thereof (e.g., cetearyl alcohol) as well as behenyl alcohol, can be used as a saturated fatty alcohol.

According to at least one embodiment, the fatty alcohol used in the composition according to the present invention is preferably chosen from octyldodecanol, hexyldecanol and mixtures thereof.

It is preferable that the (b-1) oil be selected from plant oils, synthetic ester oils, and mixtures thereof, and preferably from plant oils.

According to the present invention, the (b-1) oil may be surrounded by a plurality of the (a-1) cationic polymers or a complex of the (a-1) cationic polymers and the (a-2) monovalent non-polymeric acid or a salt thereof, or the (b-1) oil may be present in the hollow of a capsule formed by the (a-1) cationic polymers or the above complex. In other words, the (b-1) oil may be covered by the (a-1) cationic polymers or the above complex, or a capsule formed by the (a-1) cationic polymers and the above complex includes the (b-1) oil in the hollow of the capsule.

The (b-1) oil which is surrounded by the (a-1) cationic polymers or the above complex, or present in the hollow of the capsule formed by the (a-1) cationic polymers or the above complex cannot directly make contact with a keratin substance such as skin. Thus, even if the (b-1) oil has a sticky or greasy feeling of use, the composition according to the present invention would not provide a sticky or greasy feeling of use.

The amount of the (b-1) oil(s) in the composition according to the present invention may be 1% by weight or more, preferably 10% by weight or more, and more preferably 20% by weight or more, relative to the total weight of the composition.

The amount of the (b-2) oil(s) in the composition according to the present invention may be 50% by weight or less, preferably 45% by weight or less, and more preferably 40% by weight or less, relative to the total weight of the composition.

The amount of the (b-2) oil(s) in the composition according to the present invention may be from 1% to 50% by weight, preferably from 10% to 45% by weight, and more preferably from 20% to 40% by weight, relative to the total weight of the composition.

### [Fatty Acid]

The composition according to the present invention comprises (b-2) at least one fatty acid. If two or more fatty acids are used, they may be the same or different.

The term “fatty acid” here means a carboxylic acid with a long aliphatic carbon chain.

The (b-2) fatty acid has at least 4 carbon atoms, preferably at least 6 carbon atoms, and more preferably at least 8 carbon atoms. The (b-2) fatty acid may comprise up to 24 carbon atoms, preferably up to 22 carbon atoms, and more preferably up to 20 carbon atoms. It is preferable that the (b-2) fatty acid be selected from C₆-C₂₄ fatty acid, more preferably C₆-C₂₂ fatty acid, and even more preferably C₈-C₂₀ fatty acid.

The (b-2) fatty acid may be selected from saturated or unsaturated, linear or branched fatty acids. Thus, the (b-2) fatty acid may be selected from C₄-C₂₄, preferably C₆-C₂₂, more preferably C₈-C₂₀ saturated and unsaturated, linear or branched fatty acids.

As the unsaturated, linear or branched fatty acids, mono-unsaturated, linear or branched fatty acids or polyunsaturated, linear or branched fatty acids may be used. As the unsaturated moiety of the unsaturated, linear or branched fatty acids, a carbon-carbon double bond or a carbon-carbon triple bond may be mentioned.

As the saturated fatty acid, mention may be made of, for example, caprylic acid (C₈), pelargonic acid (C₉), capric acid (C₁₀), lauric acid (C₁₂), myristic acid (C₁₄), pentadecanoic acid (C₁₅), palmitic acid (C₁₆), heptadecanoic acid (C₁₇), stearic acid (C₁₈), isostearic acid (C₁₈), nonadecanoic acid (C₁₉), arachidic acid (C₂₀), behenic acid (C₂₂), and lignoceric acid (C₂₄).

As the unsaturated fatty acid, mention may be made of, for example, myristoleic acid (C₁₄), palmitoleic acid (C₁₆), oleic acid (C₁₈), linoleic acid (C₁₈), linolenic acid (C₁₈), elaidic acid (C₁₈), arachidonic acid (C₂₀), eicosenoic acid (C₂₀), erucic acid (C₂₂), and nervonic acid (C₂₄). It is preferable that the (b-2) fatty acid be selected from C₈-C₁₈ saturated or unsaturated, linear or branched fatty acids, and more preferably from the group consisting of caprylic acid, capric acid, oleic acid, linoleic acid, stearic acid, isostearic acid and mixtures thereof.

The (b-2) fatty acid may be in the form of a free acid or in the form of a salt thereof. As a salt of the fatty acid, mention may be made of an inorganic salt such as an alkali metal salt (a sodium salt, a potassium salt, or the like) and an alkaline earth metal salt (a magnesium salt, a calcium salt, or the like); and an organic salt such as an ammonium salt (a quaternary ammonium salt or the like) and an amine salt (a triethanolamine salt, a triethylamine salt, or the like). A single type of fatty acid salt or a combination of different type of fatty acid salts may be used. Further, a combination of one or more fatty acid in the form of a free acid and one or more fatty acid in the form of a salt may be used, in which one or more type of salts may also be used.

The amount of the (b-2) fatty acid(s) in the composition according to the present invention may be 0.01% by weight or more, preferably 0.05% by weight or more, and more preferably 0.1% by weight or more, relative to the total weight of the composition. It may be even more preferable that the amount of the (b-2) fatty acid(s) in the composition according to the present invention be 1% by weight or more, relative to the total weight of the composition.

On the other hand, the amount of the (b-2) fatty acid(s) in the composition according to the present invention may be 15% by weight or less, preferably 10% by weight or less, and more preferably 5% by weight or less, relative to the total weight of the composition. It may be even more preferable that the amount of the (b-2) fatty acid(s) in the composition according to the present invention be 4% by weight or less, relative to the total weight of the composition.

Accordingly, the amount of the (b-2) fatty acid(s) in the composition according to the present invention may range from 0.01% to 15% by weight, preferably from 0.05% to 10% by weight, and more preferably from 0.1% to 5% by weight, relative to the total weight of the composition. It may be even more preferable that the amount of the (b-2) fatty acid(s) in the composition according to the present invention be from 1% to 4% by weight, relative to the total weight of the composition.

[pH]

The pH of the composition according to the present invention may be from 3 to 9, preferably from 3.3 to 8.5, and more preferably from 3.5 to 8.

At a pH of from 3 to 9, the (a) particle can be very stable.

The pH of the composition according to the present invention may be adjusted by adding at least one alkaline agent and/or at least one acid, other than the (a-2) monovalent non-polymeric acid or a salt thereof. The pH of the composition according to the present invention may also be adjusted by adding at least one buffering agent.

### (Alkaline Agent)

The composition according to the present invention may comprise at least one alkaline agent. Two or more alkaline agents may be used in combination. Thus, a single type of alkaline agent or a combination of different types of alkaline agents may be used.

The alkaline agent may be an inorganic alkaline agent. It is preferable that the inorganic alkaline agent be selected from the group consisting of ammonia; alkaline metal hydroxides; alkaline earth metal hydroxides; alkaline metal phosphates and monohydrogenophosphates such as sodium phosphate or sodium monohydrogen phosphate.

As examples of the inorganic alkaline metal hydroxides, mention may be made of sodium hydroxide and potassium hydroxide. As examples of the alkaline earth metal hydroxides, mention may be made of calcium hydroxide and magnesium hydroxide. As an inorganic alkaline agent, sodium hydroxide is preferable.

The alkaline agent may be an organic alkaline agent. It is preferable that the organic alkaline agent be selected from the group consisting of monoamines and derivatives thereof; diamines and derivatives thereof; polyamines and derivatives thereof; basic amino acids and derivatives thereof; oligomers of basic amino acids and derivatives thereof; polymers of basic amino acids and derivatives thereof; urea and derivatives thereof; and guanidine and derivatives thereof.

As examples of the organic alkaline agents, mention may be made of alkanolamines such as mono-, di- and tri-ethanolamine, and isopropanolamine; urea, guanidine and their derivatives; basic amino acids such as lysine, ornithine or arginine; and diamines such as those described in the structure below:

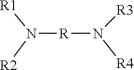

wherein R denotes an alkylene such as propylene optionally substituted by a hydroxyl or a C₁-C₄ alkyl radical, and R₁, R₂, R₃ and R₄ independently denote a hydrogen atom, an alkyl radical or a C₁-C₄ hydroxyalkyl radical, which may be exemplified by 1,3-propanediamine and derivatives thereof. Arginine, urea and monoethanolamine are preferable.

The alkaline agent(s) may be used in a total amount of from 0.01% to 15% by weight, preferably from 0.02% to 10% by weight, more preferably from 0.03% to 5% by weight, relative to the total weight of the composition, depending on their solubility.

### (Acid)

The composition according to the present invention may comprise at least one acid. Two or more acids may be used in combination. Thus, a single type of acid or a combination of different types of acids may be used.

As the acid, mention may be made of any inorganic or organic acids, preferably inorganic acids, which are commonly used in cosmetic products, such as HCl. A polyvalent acid may be used.

The acid(s) may be used in a total amount of from 0.01% to 15% by weight, preferably from 0.02% to 10% by weight, more preferably from 0.03% to 5% by weight, relative to the total weight of the composition, depending on their solubility.

### (Buffering Agent)

The composition according to the present invention may comprise at least one buffering agent. Two or more buffering agents may be used in combination. Thus, a single type of buffering agent or a combination of different types of buffering agents may be used.

As the buffering agent, mention may be made of an acetate buffer (for example, acetic acid+sodium acetate), a phosphate buffer (for example, sodium dihydrogen phosphate+di-sodium hydrogen phosphate), a citrate buffer (for example, citric acid+sodium citrate), a borate buffer (for example, boric acid+sodium borate), a tartrate buffer (for example, tartaric acid+sodium tartrate dihydrate), Tris buffer (for example, tris(hydroxymethyl)aminomethane), and a Hepes buffer (4-(2-hydroxyethyl)-1-piperazineethanesulfonic acid).

### [Optional Additives]

The composition according to the present invention may comprise, in addition to the aforementioned components, components typically employed in cosmetics, specifically, surfactants/emulsifiers, hydrophilic or lipophilic thickeners, derived from, for example, synthetic polymers other than the (a-1) cationic polymer; volatile or non-volatile organic solvents; anionic polymers; amphoteric polymers; nonionic polymers such as beta-glucan; silicones and silicone derivatives other than the (b-1) oil; natural extracts derived from animals or vegetables other than the (b-1) oil; waxes; and the like, within a range which does not impair the effects of the present invention.

The composition according to the present invention may comprise the above optional additive(s) in an amount of from 0.01% to 30% by weight, preferably from 0.05% to 20% by weight, and more preferably from 0.1% to 10% by weight, relative to the total weight of the composition.

The composition according to the present invention may include a very limited amount of surfactant(s)/emulsifier(s) and/or synthetic thickener(s) and/or organic solvent(s) in view of environmental friendliness.

The amount of the surfactant(s)/emulsifier(s) and/or synthetic thickener(s) and/or organic solvent(s) in the composition according to the present invention may be 1% by weight or less, preferably 0.1% by weight or less, and more preferably 0.01% by weight or less, relative to the total weight of the composition. It is particularly preferable that the composition according to the present invention includes no surfactant/emulsifier or synthetic thickener or organic solvent.

### [Preparation]

The composition according to the present invention can be prepared by mixing the essential ingredient(s) as explained above, and optional ingredient(s), if necessary, as explained above.

The method and means to mix the above essential and optional ingredients are not limited. Any conventional method and means can be used to mix the above essential and optional ingredients to prepare the composition according to the present invention.

The composition according to the present invention can be prepared by simple or easy mixing with a conventional mixing means such as a stirrer and a homogenizer. Also, heating may not be necessary. Therefore, the process for preparing the composition according to the present invention may be environmentally friendly.

### [Cosmetic Composition]

The composition according to the present invention may be intended to be used as a cosmetic composition. Thus, the cosmetic composition according to the present invention may be intended for application onto a keratin substance. Keratin substance here means a material containing keratin as a main constituent element, and examples thereof include the skin, scalp, nails, lips, hair, and the like. Thus, it is preferable that the cosmetic composition according to the present invention be used for a cosmetic process for the keratin substance, in particular skin.

Thus, the cosmetic composition according to the present invention may be a skin cosmetic composition, preferably a skin care composition or a skin makeup composition, and more preferably a skin care composition.

### [Form]

In the composition according to the present invention, the (b-1) oil can form fatty phases, the (a-3) water can form an aqueous phase, and the fatty phases can be dispersed in the aqueous phase. Thus, the aqueous phase can function as a continuous phase, and the fatty phase can function as a dispersed phase.

Thus, the composition according to the present invention may be in the form of an O/W dispersion such as an O/W emulsion. If the composition according to the present invention is of the O/W type, it can provide a fresh sensation due to the (a-3) water which forms the outer phase thereof.

### [Mechanism]

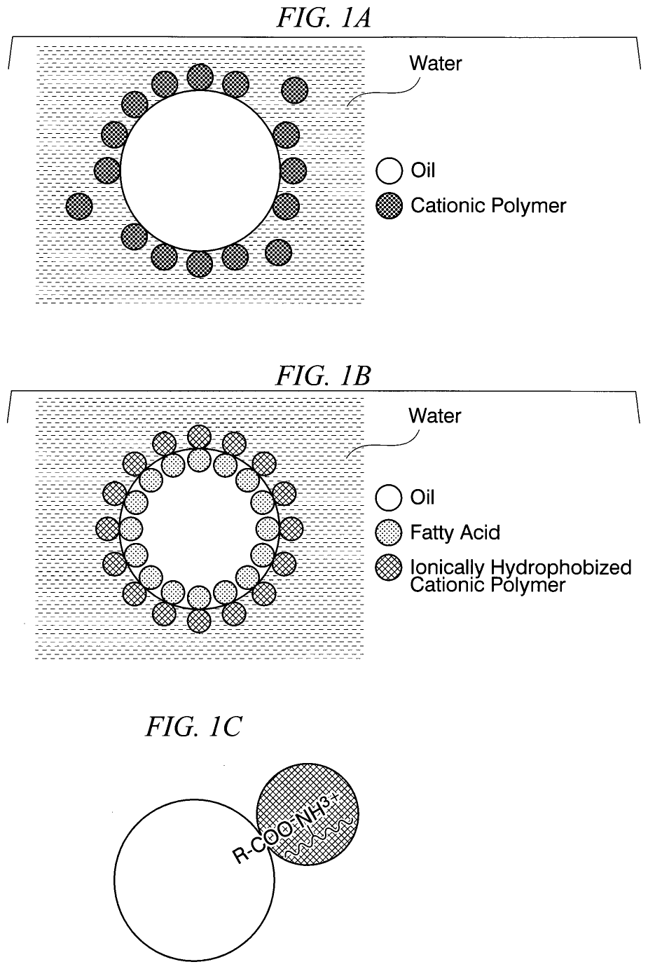

FIG. 1 shows schematic drawings showing the behaviors of the (a-1) cationic polymers around a (b) fatty phase, such as an oil droplet, dispersed in an (a) aqueous phase, and the examples of hydrophobicization of the (a-1) cationic polymer, in one embodiment of the present invention.

A plurality of the (a-1) cationic polymers or a complex of the (a-1) cationic polymers and the (a-2) monovalent non-polymeric acid or a salt thereof can be present at the interface between the (b) fatty phase and the (a) aqueous phase. Thus, the (a-1) cationic polymers or the above complex can form an emulsion without the aid by any conventional surfactant or emulsifier. The emulsion formed by the (a-1) cationic polymer or the above complex may be similar to a so-called Pickering emulsion.

Alternatively, a plurality of the (a-1) cationic polymers or the above complex can form a capsule having a hollow. The (b-1) oil can be present in the hollow. In other words, the (b-1) oil can be incorporated into the capsule. The wall of the capsule may be composed of a continuous layer or film formed from the (a-1) cationic polymers or the above complex. While not wishing to be bound by theory, it is believed that the (a-1) cationic polymers or the above complex can re-organize at the interface of the (b-1) oil and the (a-3) water to spontaneously form a capsule having a hollow to include the (b-1) oil. For example, a continuous aqueous phase comprising the (a-3) water and dispersed phases comprising the (b-1) oil in the capsule can form an O/W emulsion which may also be similar to a so-called Pickering emulsion.

The (b) fatty phase comprises the (b-2) fatty acid. The (b-2) fatty acid in the (b) fatty phase can hydrophobicize the (a-1) cationic polymer in-situ. FIG. 1 shows a scheme of hydrophobicization of the (a-1) cationic polymer by the (b-2) fatty acid FIG. 1A shows a schematic drawing showing the behaviour of the (a-1) cationic polymers around a (b) fatty phase, such as an oil droplet, dispersed in an (a) aqueous phase, in case of the (b) fatty phase including no (b-2) fatty acid.

In FIG. 1A, the (a-1) cationic polymer has insufficient hydrophobicity. Therefore, the affinity between the (a-1) cationic polymer and the (b) fatty phase including the (b-1) oil is limited. Thus, the emulsification of the (b) fatty phase in the (a) aqueous phase is less stable.

On the other hand, FIG. 1B shows a schematic drawing showing the behaviour of the (a-1) cationic polymers around a (b) fatty phase, such as an oil droplet, dispersed in an (a) aqueous phase, in case of the (b) fatty phase including (b-2) fatty acid.

In FIG. 1B, the carboxylic croup in the (b-2) fatty acid in the (b) fatty phase can ionically interact with the cationic or cationisable group, such as an ammonium group or amino group, in the (a-1) cationic polymer, as shown in FIG. 1C. Accordingly, the (a-1) cationic polymer is ionically hydrophobicized and can have sufficient hydrophobicity. Therefore, the affinity between the (a-1) cationic polymer and the (b) fatty phase including the (b-1) oil is enhanced.

Thus, the emulsification of the (b) fatty phase in the (a) aqueous phase is stable.

### [Film]

The composition according to the present invention can be used for easily preparing a film.

Thus, the present invention may also relate to a process for preparing a film, preferably a cosmetic film, optionally with a thickness of preferably more than 0.5 μm, more preferably 1.0 μm or more, and even more preferably 1.5 μm or more, comprising:
applying onto a substrate, preferably a keratin substance, more preferably skin, the composition according to the present invention; and
drying the composition.

The upper limit of the thickness of the film according to the present invention is not limited. Thus, for example, the thickness of the film according to the present invention may be 1 mm or less, preferably 500 μm or less, more preferably 300 μm or less, and even more preferably 100 μm or less.

Since the process for preparing a film according to the present invention includes the steps of applying the composition according to the present invention onto a substrate, preferably a keratin substance, and more preferably skin, and of drying the composition, the process according to the present invention does not require any spin coating or spraying, and therefore, it is possible to easily prepare even a relatively thick film. Thus, the process for preparing a film according to present invention can prepare a relatively thick film without any special equipment such as spin coaters and spraying machines.

Even if the film according to the present invention is relatively thick, it is still thin and may be transparent, and therefore, may not be easy to perceive. Thus, the film according to the present invention can be used preferably as a cosmetic film.

If the substrate is not a keratin substance such as skin, the composition according to the present invention may be applied onto a substrate made from any material other than keratin. The materials of the non-keratinous substrate are not limited. Two or more materials may be used in combination. Thus, a single type of material or a combination of different types of materials may be used. In any event, it is preferable that the substrate be flexible or elastic.

If the substrate is not a keratin substance, it is preferable that the substrate be water-soluble, because it is possible to leave the film according to the present invention by washing the substrate with water. As examples of the water-soluble materials, mention may be made of poly(meth) acrylic acids, polyethyleneglycols, polyacrylamides, polyvinyl alcohol (PVA), starch, cellulose acetates, and the like. PVA is preferable.

If the non-keratinous substrate is in the form of a sheet, it may have a thickness of more than that of the film according to the present invention, in order to ease the handling of the film attached to the substrate sheet. The thickness of the non-keratinous substrate sheet is not limited, but may be from 1 μm to 5 mm, preferably from 10 μm to 1 mm, and more preferably from 50 to 500 μm.

It is more preferable that the film according to the present invention be releasable from the non-keratinous substrate. The mode of release is not limited. Therefore, the film according to the present invention may be peeled from the non-keratinous substrate, or released by the dissolution of the substrate sheet into a solvent such as water.

The present invention may also relate to:

(1) A film, preferably a cosmetic film, optionally with a thickness of preferably more than 0.5 μm, more preferably 1.0 μm or more, and even more preferably 1.5 μm or more, prepared by a process comprising:
applying onto a substrate, preferably a keratin substance, and more preferably skin, the composition according to the present invention; and
drying the composition, and
   (2) A film, preferably a cosmetic film, optionally with a thickness of preferably more than 0.5 μm, more preferably 1.0 μm or more, and even more preferably 1.5 μm or more, comprising:
at least one cationic polymer,
at least one monovalent non-polymeric acid or a salt thereof,
at least one oil, and
at least one fatty acid.

The above explanations regarding the cationic polymer and the monovalent non-polymeric acid or a salt thereof, as well as the above oil and fatty acid, can apply to those in the above films (1) and (2).

The film thus obtained above can be self-standing. The term “self-standing” here means that the film can be in the form of a sheet and can be handled as an independent sheet without the assistance of a substrate or support. Thus, the term “self-standing” may have the same meaning as “self-supporting”.

It is preferable that the film according to the present invention be hydrophobic.

The term “hydrophobic” in the present specification means that the solubility of the film in water (preferably with a volume of 1 liter) at from 20 to 40° C., preferably from 25 to 40° C., and more preferably from 30 to 40° C. is less than 10% by weight, preferably less than 5% by weight, more preferably less than 1% by weight, and even more preferably less than 0.1% by weight, relative to the total weight of the film. It is most preferable that the film is not soluble in water.

If the film according to the present invention is hydrophobic, the film can have water-resistant properties, and therefore, it can remain on a keratin substance such as skin even if the surface of the keratin substance is wet due to, for example, sweat and rain. Thus, when the film according to the present invention provides any cosmetic effect, the cosmetic effect can last a long time.

On the other hand, the film according to the present invention can be easily removed from a keratin substance such as skin under alkaline conditions such as a pH of from 8 to 12, preferably from 9 to 11. Therefore, the film according to the present invention is difficult to remove with water, while it can be easily removed with a soap which can provide such alkaline conditions.

The film according to the present invention may comprise at least one biocompatible and/or biodegradable polymer layer. Two or more biocompatible and/or biodegradable polymers may be used in combination. Thus, a single type of biocompatible and/or biodegradable polymer or a combination of different types of biocompatible and/or biodegradable polymers may be used.

The term “biocompatible” polymer in the present specification means that the polymer does not have excess interaction between the polymer and cells in the living body including the skin, and the polymer is not recognized by the living body as a foreign material.

The term “biodegradable” polymer in the present specification means that the polymer can be degraded or decomposed in a living body due to, for example, the metabolism of the living body itself or the metabolism of the microorganisms which may be present in the living body. Also, the biodegradable polymer can be degraded by hydrolysis.

If the film according to the present invention includes a biocompatible and/or biodegradable polymer, it is less irritable or not irritable to the skin, and does not cause any rash. In addition, due to the use of a biocompatible and/or biodegradable polymer, the cosmetic sheet according to the present invention can adhere well to the skin.

The film according to the present invention can be used for cosmetic treatments of keratin substances, preferably skin, in particular the face. The film according to the present invention can be in any shape or form. For example, it can be used as a full-face mask sheet, or a patch for a part of the face such as the cheek, nose, and around the eyes.

If the film according to the present invention includes at least one hydrophilic or water-soluble UV filter, it can provide UV shielding effects derived from the hydrophilic or water-soluble UV filter. Normally, a hydrophilic or water-soluble UV filter can be removed from the surface of a keratinous substrate such as skin by water such as sweat and rain. However, since the hydrophilic or water-soluble UV filter is included in the film according to the present invention, it is difficult for the hydrophilic or water-soluble UV filter to be removed by water, thereby resulting in long-lasting UV shielding effects.

### [Cosmetic Process and Use]

The present invention also relates to:
a cosmetic process for a keratin substance such as skin, comprising: applying to the keratin substance the composition according to the present invention; and drying the composition to form a cosmetic film on the keratin substance; or
a use of the composition according to the present invention for the preparation of a cosmetic film on a keratin substance such as skin.

The cosmetic process here means a non-therapeutic cosmetic method for caring for and/or making up the surface of a keratin substance such as skin.

In both the above process and use, the above cosmetic film is resistant to water with a pH of 7 or less, and is removable with water with a pH of more than 7, preferably 8 or more, and more preferably 9 or more.

In other words, the above cosmetic film can be water-resistant under neutral or acidic conditions such as a pH of 7 or less, preferably in a range of 6 or more and 7 or less, and more preferably in a range of 5 or more and 7 or less, while the above cosmetic film can be removed under alkaline conditions such as a pH of more than 7, preferably 8 or more, and more preferably 9 or more. The upper limit of the pH is preferably 13, more preferably 12, and even more preferably 11.

Accordingly, the above cosmetic film can be water-resistant, and therefore, it can remain on a keratin substance such as skin even if the surface of the keratin substance is wet due to, for example, sweat and rain. On the other hand, the above cosmetic film can be easily removed from a keratin substance such as skin under alkaline conditions. Therefore, the film according to the present invention is difficult to remove with water, while it can be easily removed with soap which can provide alkaline conditions.

If the above cosmetic film includes a UV filter which may be present in the composition according to the present invention, the above cosmetic film can protect a keratin substance such as skin from UV rays, thereby limiting the darkening of the skin, improving the colour and uniformity of the complexion, and/or treating aging of the skin.

Furthermore, the above cosmetic film may have cosmetic effects such as capturing sebum, matting the appearance of a keratin substrate such as skin, absorbing or adsorbing malodour, and/or protecting the keratin substance from, for example, dirt or pollutant, due to the properties of the cosmetic film, even if the cosmetic film does not include any cosmetic active ingredient.

In addition, the above cosmetic film may immediately change or modify the appearance of the skin by changing light reflection on the skin and the like, even if the cosmetic film does not include any cosmetic active ingredient. Therefore, it may be possible for the above cosmetic film to conceal skin defects such as pores or wrinkles. Further, the above cosmetic film may immediately change or modify the feel to the touch of the skin by changing the surface roughness on the skin and the like. Furthermore, the above cosmetic film may immediately protect the skin by covering the surface of the skin and shielding the skin, as a barrier, from environmental stresses such as pollutants, contaminants and the like.

The above cosmetic effects can be adjusted or controlled by changing the chemical composition, the thickness and/or the surface roughness of the above cosmetic film.

If the above cosmetic film includes at least one additional cosmetic active ingredient other than the (b-1) oil, the cosmetic film can have cosmetic effects provided by the additional cosmetic active ingredient(s). For example, if the cosmetic film includes at least one cosmetic active ingredient selected from anti-aging agents, anti-sebum agents, deodorant agents, anti-perspirant agents, whitening agents and a mixture thereof, the cosmetic film can treat the aging of the skin, absorbing sebum on the skin, controlling odors on the skin, controlling perspiration on the skin, and/or whitening of the skin.

It is also possible to apply a makeup cosmetic composition onto the cosmetic film or sheet according to the present invention after it has been applied onto the skin.

The present invention also relates to a use of (b-2) at least one fatty acid in a composition, comprising:
(a-1) at least one cationic polymer;
(a-2) at least one monovalent non-polymeric acid or a salt thereof;
(a-3) water; and
(b-1) at least one oil,
   in order to increase the amount of the (b-1) oil in the composition to be 1% by weight or more, preferably 10% by weight or more, and more preferably 20% by weight or more, relative to the total weight of the composition.

The above explanations regarding the cationic polymer and the monovalent non-polymeric acid or a salt thereof, as well as the above oil and fatty acid, can apply to those in the above use.

The above use according to the present invention can enhance the stability of the composition even though the composition comprises a relatively large amount of oil, such as 1% by weight or more, preferably 10% by weight or more, and more preferably 20% by weight or more, relative to the total weight of the composition. Therefore, the phase separation of the composition can be prevented for a long period of time.

### EXAMPLES

The present invention will be described in a more detailed manner by way of examples. However, they should not be construed as limiting the scope of the present invention.

### Examples 1-5 and Comparative Example 1

### [Preparations]

### Example 1

Two grams of an aqueous solution (80 wt %) of chitosan (M.W.: 100,000) was added to water, followed by adding 1 g of lactic acid while stirring. Next, 20 g of Zea mays (corn) germ oil and 0.5 g of oleic acid were added while stirring. Thereby, an O/W type composition comprising a chitosan-lactate complex was prepared. The preparation was performed at room temperature without heating.

### Examples 2-5

In Examples 2-5, the procedures to prepare the composition according to Example 1 were repeated with the proviso that the ingredients shown in Table 1 were used to prepare the compositions according to Examples 2-5.

### Comparative Example 1

In Comparative Example 1, the procedures to prepare the composition according to Example 1 was repeated with the proviso that oleic acid was not used.

The ingredients used to prepare the compositions according to Examples 1-5 and Comparative Example 1 are shown in Table 1. The units of the amounts of the ingredients shown in Table 1 are all “grams”.

TABLE 1

| | | | | | | Comp. |
|---|---|---|---|---|---|---|
| | Ex. 1 | Ex. 2 | Ex. 3 | Ex. 4 | Ex. 5 | Ex. 1 |
| Water | qsp 100 | qsp 100 | qsp 100 | qsp 100 | qsp 100 | qsp 100 |
| Chitosan | 2 | 2 | 2 | 2 | 2 | 2 |
| (80 wt % | | | | | | |
| aqueous | | | | | | |
| solution) | | | | | | |
| Zea Mays | 20 | 20 | 20 | 20 | — | 20 |
| (Corn) | | | | | | |
| Germ Oil | | | | | | |
| Diisopropyl | — | — | — | — | 20 | — |
| Sebacate | | | | | | |
| Oleic Acid | 0.5 | 1.15 | 1.7 | 3.5 | 0.5 | — |
| Lactic Acid | 1 | 1 | 1 | 1 | 1 | 1 |
| Stability | Very | Very | Very | Very | Good | Very |
| | Good | Good | Good | Good | | Poor |

### [Evaluations]

### (Microscopic Observation)

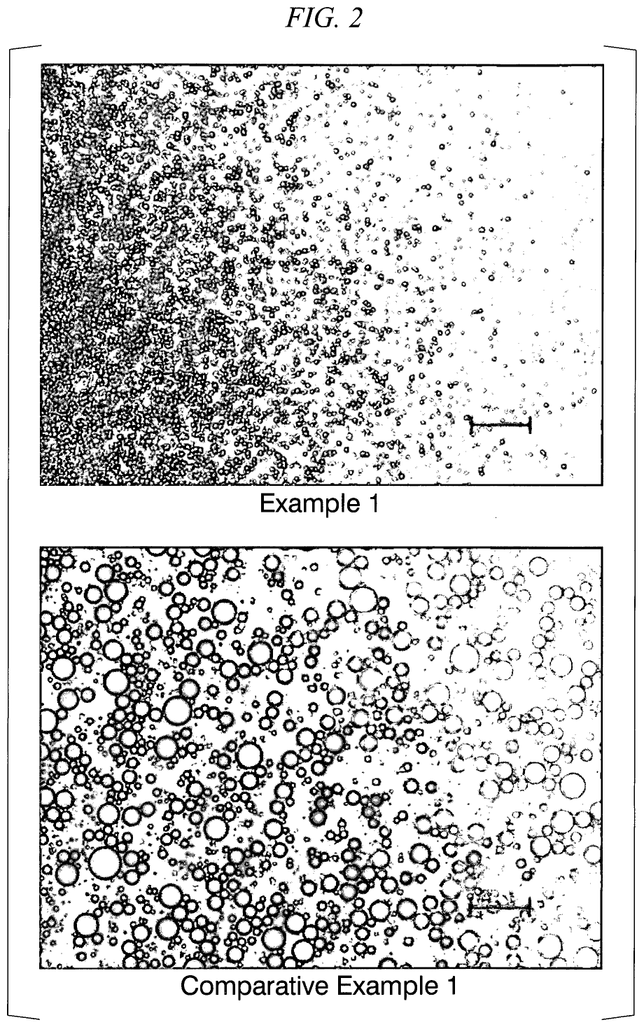

Each of the composition according to Example 1 and the composition according to Comparative Example 1 was subjected to microscopic observation. The photomicrographs of the compositions according to Example 1 and Comparative Example 1 are shown in FIG. 2.

The size of the oil droplets in the composition according to Example 1 was small, and the oil droplets were uniformly dispersed, while the size of the oil droplets in the composition according to Comparative Example 1 vary, and the oil droplets were not uniformly dispersed, forming some agglomerates.

### (Stability)

Each of the compositions according to Examples 1-5 and Comparative Example 1 was stored in a transparent vessel at room temperature for one day. The stability of the compositions was visually observed and evaluated in accordance with the following criteria.

Very Good: The composition was uniform.

Good: The composition was uniform, but slight unevenness was observed.

Poor: The composition was not uniform, and unevenness was observed.

Very Poor: Phase separation was observed.

The results are shown in Table 1.

The compositions according to Examples 1-5 were stable to maintain emulsified uniform appearance, without phase separation, while the composition according to Comparative Example 1 was not stable because phase separation was observed.

The compositions according to Examples 1-4 were more preferable because they showed better stability than the composition according to Example 5.

### Examples 6-7 and Comparative Example 2

### [Preparations]

### Example 6

Two grams of an aqueous solution (80 wt %) of chitosan (M.W.: 100,000) was added to water, followed by adding 1 g of lactic acid while stirring. Next, 30 g of Zea mays (corn) germ oil and 1.7 g of oleic acid were added while stirring. Thereby, an O/W type composition comprising a chitosan-lactate complex was prepared. The preparation was performed at room temperature without heating.

### Examples 7

In Example 7, the procedures for preparing the composition according to Example 6 were repeated with the proviso that the amount of Zea mays (corn) germ oil was 40 g.

### Comparative Example 2

In Comparative Example 2, the procedures for preparing the composition according to Example 6 was repeated with the proviso that the amount of Zea mays (corn) germ oil was 51 g.

The ingredients used to prepare the compositions according to Examples 6-7 and Comparative Example 2 are shown in Table 2. The units of the amounts of the ingredients shown in Table 2 are all “grams”.

TABLE 2

| | Ex. 6 | Ex. 7 | Comp. Ex. 2 |
|---|---|---|---|
| Water | qsp 100 | qsp 100 | qsp 100 |
| Chitosan | 2 | 2 | 2 |
| (80 wt % aqueous solution) | | | |
| Zea Mays (Corn) Germ Oil | 30 | 40 | 51 |
| Oleic Acid | 1.7 | 1.7 | 1.7 |
| Lactic Acid | 1 | 1 | 1 |
| Stability | Very | Very | Very |
| | Good | Good | Poor |

### [Evaluations]

### (Stability)

Each of the compositions according to Examples 6-7 and Comparative Example 2 was stored in a transparent vessel at room temperature for one day. The stability of the compositions was visually observed and evaluated in accordance with the following criteria.

Very Good: The composition was uniform.

Good: The composition was uniform, but slight unevenness was observed.

Poor: The composition was not uniform, and unevenness was observed.

Very Poor: Phase separation was observed.

The results are shown in Table 2.

The compositions according to Examples 6-7 were stable to maintain emulsified uniform appearance, without phase separation, while the composition according to Comparative Example 2 was not stable because phase separation was observed.

### Examples 8-9

### [Preparations]

### Example 8

Two grams of an aqueous solution (80 wt %) of chitosan (M.W.: 100,000) was added to water, followed by adding 1 g of lactic acid while stirring. Next, 30 g of Zea mays (corn) germ oil and 1.7 g of octanoic acid were added while stirring. Thereby, an O/W type composition comprising a chitosan-lactate complex was prepared. The preparation was performed at room temperature without heating.

### Examples 9

In Example 9, the procedures for preparing the composition according to Example 8 were repeated with the proviso that linoleic acid. was used instead of octanoic acid.

The ingredients used to prepare the compositions according to Examples 8-9 are shown in Table 3. The units of the amounts of the ingredients shown in Table 3 are all “grams”.

TABLE 3

| | Ex. 8 | Ex. 9 |
|---|---|---|
| Water | qsp 100 | qsp 100 |
| Chitosan | 2 | 2 |
| (80 wt % aqueous solution) | | |
| Zea Mays (Corn) Germ Oil | 30 | 40 |
| Octanoic Acid | 1.7 | — |
| Linoleic Acid | — | 1.7 |
| Lactic Acid | 1 | 1 |
| Stability | Very | Very |
| | Good | Good |

### [Evaluations]

### (Stability)

Each of the compositions according to Examples 8-9 was stored in a transparent vessel at room temperature for one day. The stability of the compositions was visually observed and evaluated in accordance with the following criteria.

Very Good: The composition was uniform.

Good: The composition was uniform, but slight unevenness was observed.

Poor: The composition was not uniform, and unevenness was observed.

Very Poor: Phase separation was observed.

The results are shown in Table 3.

The compositions according to Examples 8-9 were stable to maintain emulsified uniform appearance, without phase separation.

1. A composition, comprising:
(a-1) at least one cationic polymer;
(a-2) at least one monovalent non-polymeric acid or a salt thereof;
(a-3) water;
(b-1) at least one oil; and
(b-2) at least one fatty acid.

2. The composition according to claim 1, wherein the (a-1) cationic polymer is hydrophobicized by the (b-2) fatty acid.

3. The composition according to claim 1, wherein the (a-1) cationic polymer is selected from the group consisting of cyclopolymers of alkyldiallylamine and cyclopolymers of dialkyldiallylammonium such as (co)polydiallyldialkyl ammonium chloride, (co)polyamines such as (co)polylysines and chitosans, cationic (co)polyaminoacids such as collagen, cationic cellulose polymers, and salts thereof.

4. The composition according to claim 1, wherein the amount of the (a-1) cationic polymer(s) in the composition is from 0.01% to 15% by weight, preferably from 0.05% to 10% by weight, and more preferably from 0.1% to 5% by weight, relative to the total weight of the composition.

5. The composition according to claim 1, wherein the (a-2) monovalent non-polymeric acid is a monovalent non-polymeric organic acid, preferably a monovalent carboxylic acid, and more preferably lactic acid.

6. The composition according to claim 1, wherein the amount of the (a-2) monovalent non-polymeric acid or a salt thereof in the composition is from 0.01% to 20% by weight, preferably from 0.05% to 15% by weight, and more preferably from 0.1% to 10% by weight, relative to the total weight of the composition.

7. The composition according to claim 1, wherein the amount of the (a-3) water in the composition is from 40% to 90% by weight, preferably from 45% to 85% by weight, and more preferably from 50% to 80% by weight, relative to the total weight of the composition.

8. The composition according to claim 1, wherein the (b-1) oil is selected from plant oils, synthetic ester oils, and mixtures thereof, and preferably from plant oils.

9. The composition according to claim 1, wherein the amount of the (b-1) oil(s) in the composition is from 1% to 50% by weight, preferably from 10% to 45% by weight, and more preferably from 20% to 40% by weight, relative to the total weight of the composition.

10. The composition according to claim 1, wherein the (b-2) fatty acid is selected from C₄-C₂₂, preferably C₆-C₂₀, more preferably C₈-C₁₈ saturated and unsaturated, linear or branched fatty acids.

11. The composition according to claim 1, wherein the amount of the (b-2) fatty acid(s) in the composition is from 0.01% to 15% by weight, preferably from 0.05% to 10% by weight, and more preferably from 0.1% to 5% by weight, relative to the total weight of the composition.

12. The composition according to claim 1, wherein the composition comprises an (a) aqueous phase comprising the (a-1) cationic polymer, the (a-2) monovalent non-polymeric acid or a salt thereof, and the (a-3) water.

13. The composition according to claim 1, wherein the composition comprises (b) fatty phases comprising the (b-1) oil, and the (b-2) fatty acid.

14. A cosmetic process for a keratin substance such as skin, comprising:
applying to the keratin substance the composition according to claim 1; and
drying the composition to form a cosmetic film on the keratin substance.

15. A use of (b-2) at least one fatty acid in a composition, comprising:
(a-1) at least one cationic polymer;
(a-2) at least one monovalent non-polymeric acid or a salt thereof;
(a-3) water; and
(b-1) at least one oil,
in order to increase the amount of the (b-1) oil in the composition to be 1% by weight or more, preferably 10% by weight or more, and more preferably 20% by weight or more, relative to the total weight of the composition.
